(19) United States
(12) Patent Application Publication
(10) Pub. No.: US 2022/0296666 A1
(43) Pub. Date: Sep. 22, 2022

(54) COMPOSITIONS AND METHODS OF PRODUCING DE-FLAVORED PEPPERCORN

(71) Applicant: BrightSeed, Inc., South San Francisco, CA (US)

(72) Inventors: Alexandra Marcela Herrmann, South San Francisco, CA (US); Swetha Mahadevan, South San Francisco, CA (US)

(21) Appl. No.: 17/835,407

(22) Filed: Jun. 8, 2022

Related U.S. Application Data
(63) Continuation of application No. PCT/US2020/063825, filed on Dec. 8, 2020.
(60) Provisional application No. 62/946,108, filed on Dec. 10, 2019.

(51) Int. Cl. A61K 36/67; A23L 27/10; A23L 5/10; A23L 3/28

(52) U.S. Cl. CPC A61K 36/67; A23L 27/10; A23L 5/12; A23L 3/28; A61K 2236/00

(57) ABSTRACT

Disclosed herein are compositions and methods for de-flavoring a peppercorn, whole or ground, that minimize piperine, a naturally occurring alkaloid imparting pungency, that minimize volatile oils imparting aroma, and that retain most of the naturally occurring metabolic micronutrients. A method is also disclosed that includes contacting a peppercorn with one or more oils thereby producing a de-flavored peppercorn.

### CROSS REFERENCE TO RELATED APPLICATIONS

This application is a continuation of PCT Application No. PCT/US2020/063825, filed Dec. 8, 2020, which claims the benefit of priority of U.S. Provisional Application No. 62/946,108, filed Dec. 10, 2019. All of the foregoing applications are incorporated herein by reference in their entireties for all purposes.

### FIELD

The present disclosure relates generally to the field of food and beverage supplements to improve, maintain, and/or promote good metabolism, digestion, and insulin response.

### BACKGROUND

### Description of the Related Art

Peppercorn is known for its delightful flavor, pungency and beneficial pharmacological properties. While the volatile oils are responsible for aroma, the primary compound attributed to impart pungency and the biting taste of peppercorns is a naturally occurring alkaloid, known as piperine. Piperine is extensively reported to offer therapeutic effects due to it antioxidant, anti-inflammatory and bioavailability enhancing properties (Gorgani et. al, 2017, Comp. Rev. of Food Sci. and Food Safety, pp 124-140).

Black peppercorns as a source of micronutrients and the role of metabolic micronutrients in regulating fat metabolism has not been explored in the past. To achieve efficacious doses of such metabolic micronutrients in the diet, however, the serving size of standard commercial black pepper that must be ingested is highly limited due to elevated levels of piperine, the primary alkaloid responsible for pungency in peppercorns. Hence, there is a need to de-flavor black pepper to yield optimal compositions of metabolic micronutrients and piperine in black pepper (whole and ground) that minimize pungency, while promoting the metabolic health benefits and bioavailability of the metabolic micronutrients.

### SUMMARY

Disclosed herein are methods and compositions useful for producing a de-flavored peppercorn. In some embodiments, the methods described herein produce a de-flavored peppercorn that removes the heat associated with peppercorn.

Some embodiments pertain to a method of producing a de-flavored peppercorn. In some embodiments, the method comprises contacting a peppercorn with an oil. In some embodiments, the method comprises pretreating peppercorn. In some embodiments, the method comprises extracting piperine from peppercorn. In some embodiments, the method comprises contacting the peppercorn with supercritical carbon dioxide. In some embodiments, the method comprises contacting peppercorn with an organic solvent. In some embodiments, the method comprises contacting peppercorn with high intensity light. In some embodiments, the method comprises contacting peppercorn with UV radiation.

Some embodiments relate to a method of preparing a de-flavored peppercorn. In some aspects, the method comprises contacting a peppercorn with supercritical carbon dioxide at a temperature from about 30° C. to about 90° C. and a pressure from about 5 to about 80 MPa, thereby producing a de-flavored peppercorn. In some embodiments, the temperature is from about 30° C. to about 60° C. In some embodiments, the pressure is from about 7.4 to about 36 MPa. In some embodiments, the peppercorn moisture content is 0 to 25% w/w. In some embodiments, the method further comprises contacting the peppercorn with a solvent. In some embodiments, the de-flavored peppercorn piperine content is about 0% to about 60% w/w of the original piperine content of the peppercorn. In some embodiments, the de-flavored peppercorn comprises 0% to 5% w/w piperine, 5 to 25% w/w moisture content, and 0.005-0.2% w/w metabolic micronutrients. In some embodiments, the de-flavored peppercorn comprises 0.01% to about 0.6% w/w piperine, greater than 60% w/w carbohydrates, less than 1% w/w fat, less than 10% w/w ash, about 10 to about 15% w/w protein, and greater than 0.05% w/w metabolic micronutrients. In some embodiments, the de-flavored peppercorn comprises 0.3% w/w piperine. In some embodiments, the metabolic micronutrients comprise at least one of N-trans-caffeoyltyramine, N-trans-feruloyltyramine, and p-coumaroyltyramine. In some embodiments, the method further comprises milling the peppercorn. In some embodiments, contacting the peppercorn with supercritical carbon dioxide is for 15 minutes to 4 hours. In some embodiments, the peppercorn is selected from a whole peppercorn, a ground peppercorn, and a crushed peppercorn.

Some embodiments relate to a dietary supplement, food ingredient or additive, a medical food, or nutraceutical. In some embodiments, the dietary supplement, food ingredient or additive, a medical food, or nutraceutical comprises a de-flavored peppercorn, and a carrier, wherein the de-flavored peppercorn comprises about 0.01% to about 0.6% w/w piperine, greater than 60% w/w carbohydrates, less than 1% w/w fat, less than 10% w/w ash, about 10 to about 15% w/w protein, and greater than 0.05% w/w metabolic micronutrients. In some embodiments, the carrier is selected from at least one of a sugar, a starch, cellulose, powdered tragacanth, malt, gelatin, talc, excipient, oil, glycol, polyol, ester, agar, buffering agent, alginic acid, isotonic saline, ethyl alcohol, pH buffered solutions, phospholipids, emulsifiers or polyesters, monoglycerides, diglycerides and medium chain triglycerides. In some embodiments, the dietary supplement, food ingredient or additive, a medical food, or nutraceutical further comprises a preservative. In some embodiments, the preservative is selected from at least one of sodium benzoate, methyl paraben, propyl paraben, sodium nitrite, sulphur dioxide, sodium sorbate, potassium sorbate or salts of edetate. In some embodiments, the metabolic micronutrients comprise at least one of N-trans-caffeoyltyramine, N-trans-feruloyltyramine, and p-coumaroyltyramine. In some embodiments, the dietary supplement, food ingredient or additive, a medical food, or nutraceutical further comprises a secondary agent. In some embodiments, the secondary agent is selected from a phytonutrient, a flavoring agent, a coating layer, a stabilizer, a colorant, a diluent, a filler, one or more alkaloid, one or more vitamin, or one or more tyramine containing hydroxycinnamic acid amides. In some embodiments, the de-flavored peppercorn is formulated to provide increased metabolic nutrients. In some embodiments, the de-flavored peppercorn further comprises 5 to 25% w/w moisture content.

Some embodiments relate to a method of producing a de-flavored peppercorn, the method comprises contacting a peppercorn with one or more oils from about 50 to about 190° C., at about 10 to about 30 psi for about 5 to about 120 minutes, thereby producing a de-flavored peppercorn. In some embodiments, the peppercorn is selected from a whole peppercorn, a ground peppercorn, and a crushed peppercorn. In some embodiments, the one or more oils is a vegetable oil. In some embodiments, the vegetable oil is selected from the group comprising of sunflower oil, safflower oil, olive oil, soybean oil, canola oil, palm oil, coconut oil, cottonseed oil, rice bran oil, a peanut oil, or a combination thereof. In some embodiments, the oil is at a temperature from about 50° C. to about 135° C. In some embodiments, the pressure is from about 14.5 psi to about 30 psi. In some embodiments, the oil is at a pH from about 3 to about 8. In some embodiments, the method further comprises agitating the peppercorn for about 5 minutes to about 120 minutes. In some embodiments, the peppercorn is fried, baked or pressure cooked. In some embodiments, the de-flavored peppercorn comprises greater than 100 mg/Kg of metabolic micronutrients. In some embodiments, the de-flavored peppercorn comprises about 0 to about 60% original piperine levels. In some embodiments, the method further comprises separating the vegetable oil from the peppercorn. In some embodiments, the method further comprises washing the peppercorn with an aqueous solution. In some embodiments, the washing removes residual vegetable oil. In some embodiments, the method further comprises drying the peppercorn. In some embodiments, the method further comprises milling the peppercorn.

Some embodiments relate to a method of producing a de-flavored peppercorn, the method comprises pretreating a peppercorn, wherein, pretreating comprises applying pressure to the peppercorn, increasing temperature applied to the peppercorn, irradiating the peppercorn, applying a solvent to the peppercorn, or a combination thereof, thereby producing a de-flavored peppercorn. In some embodiments, the pretreating comprises submerging the peppercorn in a solvent. In some embodiments, the solvent is water or a vegetable oil. In some embodiments, the method further comprises irradiating the peppercorn. In some embodiments, the irradiating is microwave irradiation. In some embodiments, the microwave irradiation is at 915 MHz. In some embodiments, the method further comprises treating the peppercorn with an enzyme. In some embodiments, the enzyme is selected from a cellulase, hemicellulase, amylase, pectinase, or a xylanase. In some embodiments, the method further comprises blanching the peppercorn. In some embodiments, the blanching is at about 40° C. to about 90° C. In some embodiments, the method further comprises treating the peppercorn with an acidic solution. In some embodiments, the acidic solution has a pH from about 2 to about 7. In some embodiments, the method further comprises treating the peppercorn with ultrasound. In some embodiments, the ultrasound is greater than 25 kHz. In some embodiments, the method further comprises extracting piperine from the peppercorn.

Some embodiments relate to a method of extracting piperine from a peppercorn. In some embodiments, the method comprises contacting a peppercorn with an organic solvent. In some embodiments, the organic solvent is an alkyl solvent or a cycloalkyl solvent. In some embodiments, the alkyl solvent is hexane or heptane.

Some embodiments relate to a method of extracting piperine from a peppercorn. In some embodiments, the method comprises contacting a peppercorn with a high intensity light. In some embodiments, the high intensity light is from about 3000 lux to about 10000 lux. In some embodiments, the method comprises subjecting a peppercorn with UV radiation. In some embodiments, the UV radiation is from about 100 nm to about 400 nm.

Some embodiments relate to a de-flavored peppercorn. In some embodiments, the de-flavored peppercorn comprises 0.01% to 6% w/w piperine; 5 to 25% w/w moisture content; 0.005-0.2% w/w metabolic micronutrients; and a carrier. In some embodiments, the de-flavored peppercorn comprises 1 to 3% piperine. In some embodiments, the moisture content is 25%.

Not all objectives mentioned in this specification are achieved in all embodiments disclosed and/or claimed herein.

### DETAILED DESCRIPTION

De-flavored peppercorn and associated methods of making de-flavored peppercorn are provided.

It is an object of some embodiments to provide a method for producing a de-flavored peppercorn. An object of some embodiments is a method to minimally process peppercorn using food safe solvents. Another object of some embodiments is to reduce the amount of piperine in peppercorn.

One or more than one of the aforementioned objects is provided by or achieved by the various compositions, methods, and uses as described herein.

### Definitions

As used herein, “peppercorn” is a broad term, and is to be given its ordinary and customary meaning to a person of ordinary skill in the art (and is not to be limited to a special or customized meaning), and refers without limitation to any peppercorn as described herein. Peppercorn includes, but is not limited to, black peppercorn, white peppercorn, green peppercorn, ground peppercorn, crushed peppercorn, brined peppercorn, whole peppercorn, fresh peppercorn, black pepper and dried peppercorn.

As used herein, a “carrier” or “excipient” as used herein are broad terms, and each is to be given its ordinary and customary meaning to a person of ordinary skill in the art (and is not to be limited to a special or customized meaning), and refers without limitation to a compound that facilitates the incorporation of a compound into cells or tissues.

As used herein, a “nutraceutical” generally refers to natural, bioactive chemical compounds that provide physiological benefits, including disease prevention and health promotion which may be used to supplement the diet. Nutraceuticals can be either purified or concentrated by using bioengineering methods and can be enhanced through genetic methods, which contain elevated levels of natural substances. Examples of nutraceuticals include isolated nutrients and herbal products and generally contain at least one of the following ingredients: a vitamin, a mineral, an herb or other botanical, an amino acid, a metabolite, constituent, extract, or combination of these ingredients. Common examples of nutraceuticals include beta-carotene, ephedra, Ginko biloba, goldenseal, valerian, ginseng, green tea extract, and echinacea. The nutraceuticals described herein may be useful for maintenance and support of, for example, healthy joints, skin, eye and brain function, heart and circulatory system, and general health.

As used herein, “metabolic micronutrients” refers to a mixture compounds that are inherently present in a peppercorn and/or a black pepper that modulate one or more physiological processes in a human or animal species. Metabolic micronutrients included, but are not limited to primary and secondary metabolites of plant biochemical pathways, small molecules, vitamins, minerals, amino acids, peptides, proteins, carbohydrates, lipids, fatty acids, sugars, starches, fibers, small molecules, and primary and secondary metabolites.

As used herein, “solvent” is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, and water, among others.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

Any percentages, ratios or other quantities referred to herein are on a weight basis, unless otherwise indicated.

### De-Flavored Peppercorn & Formulations

An aspect of the disclosure includes a de-flavored peppercorn. In some embodiments, the de-flavored peppercorn is selected from a whole peppercorn, a ground peppercorn, and a crushed peppercorn. In some embodiments, the peppercorn is black pepper. In some embodiments, the peppercorn is white peppercorn. In some embodiments, the peppercorn is green peppercorn. In some embodiments, the de-flavored peppercorn comprises 0, 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0% w/w piperine, or ranges including, between, and/or spanning the aforementioned values. In some embodiments, the de-flavored peppercorn comprises about 0% to about 6% w/w piperine. In some embodiments, the de-flavored peppercorn comprises 0% w/w piperine. In some embodiments, the de-flavored peppercorn comprises 0.1% w/w piperine. In some embodiments, the de-flavored peppercorn comprises 0.3% w/w piperine. In some embodiments, the de-flavored peppercorn comprises 0.5% w/w piperine. In some embodiments, the de-flavored peppercorn comprises 0.8% w/w piperine. In some embodiments, the de-flavored peppercorn comprises 1.0% w/w piperine. In some embodiments, the de-flavored peppercorn comprises 1.5% w/w piperine. In some embodiments, the de-flavored peppercorn comprises 2.0% w/w piperine. In some embodiments, the de-flavored peppercorn comprises 2.5% w/w piperine. In some embodiments, the de-flavored peppercorn comprises 3.0% w/w piperine. In some embodiments, the de-flavored peppercorn comprises 3.5% w/w piperine. In some embodiments, the de-flavored peppercorn comprises 4.0% w/w piperine. In some embodiments, the de-flavored peppercorn comprises 4.5% w/w piperine. In some embodiments, the de-flavored peppercorn comprises 5.0% w/w piperine. In some embodiments, the de-flavored peppercorn comprises 5.5% w/w piperine. In some embodiments, the de-flavored peppercorn comprises 6.0% w/w piperine.

In some embodiments, the de-flavored peppercorn retains the natural micronutrients with a lowered piperine level. In some embodiments, the de-flavored peppercorn comprises 1, 10, 20, 30, 40, 50, 60, 70, 80, 90, 100, 150, 200, 250, 300, 350, 400, 450, 500, 550, 600, 650, 700, 750, 800, 850, 900, 950, 1000, 2000 mg/Kg of metabolic micronutrients, or ranges including, between, and/or spanning the aforementioned values. In some embodiments, the de-flavored peppercorn further comprises the addition of one or more micronutrients. In some embodiments, the one or more micronutrients includes manganese, vitamin A, vitamin B, vitamin C, vitamin K, copper, fiber, iron, chromium, calcium, protein, carbohydrates, fat, fiber, sugars, or starch. In some embodiments, the metabolic micronutrients include N-trans-caffeoyltyramine, N-cis-caffeoyltyramine, N-trans-feruloyltyramine, N-cis-feruloyltyramine, p-coumaroyltyramine, or a combination thereof.

In some embodiments, the de-flavored peppercorn includes about 0%, 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25% w/w moisture content, or ranges including, between, and/or spanning the aforementioned values.

In some embodiments, the de-flavored peppercorn of this disclosure may further comprise one or more acceptable excipients and/or carriers for oral consumption. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, phospholipid, sodium starch glycolate, crospovidone, sucrose, vegetable gums, lactose, methylcellulose, povidone, carboxymethylcellulose, corn starch, cyclodextrins, chitosan and its derivatives and the like (including mixtures thereof). In some embodiments, the carrier includes at least one of calcium carbonate, magnesium stearate, maltodextrin, and mixtures thereof. The various ingredients and the excipient and/or carrier are mixed and formed into the desired form using conventional techniques.

In some embodiments, the de-flavored peppercorn may further comprise a nutraceutically acceptable excipient or nutraceutically acceptable carrier. In some embodiments, the nutraceutically acceptable excipient or nutraceutically acceptable carrier is selected from medium chain triglycerides, diglycerides and monoglycerides, caprylic acid, linoleic acid, linoleic acid, oleic acid, keto-oleic acid. In some embodiments, the nutraceutically acceptable excipient or nutraceutically acceptable carrier further comprises a second fatty acid component having one or more non-activated fatty acid selected from linoleic acid, α-linoleic acid, γ-linoleic acid, oleic acid, eicosapentaenoic acid (EPA), docosahexaenoic acid (DEIA), or derivatives thereof.

In some embodiments, the de-flavored peppercorn may further include one or more secondary agents selected from vitamin A, vitamin B, vitamin B-1, vitamin B-2, vitamin B-6, vitamin B-12, vitamin C, vitamin D, vitamin D3, vitamin E, selenium, β-carotene, Ginkgo biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof. In some embodiments, the de-flavored peppercorn may include one or more secondary agent selected from policosanols, phospholipid, guggulipids, rice bran extract, wheat germ, wheat germ extract, beeswax, and red yeast rice extract, and such the de-flavored peppercorn may be formulated to promote a healthy heart and circulatory system. In other embodiments, the de-flavored peppercorn may include one or more secondary agent selected from vitamin B-1, vitamin B-2, vitamin B-6, vitamin B-12, vitamin C, vitamin D, vitamin D3, vitamin E, selenium, goldenseal, valerian, ginseng, and echinacea and such the de-flavored peppercorn may be formulated to promote healthy cell proliferation. In still other embodiments, the de-flavored peppercorn may include one or more secondary agent selected from vitamin A, vitamin C, vitamin E, and n-carotene, and such the de-flavored peppercorn may be formulated to promote healthy eyes. In yet other embodiments, the de-flavored peppercorn may include one or more secondary agent selected from vitamin A, vitamin C, vitamin E, selenium, Ginkgo biloba, goldenseal, valerian, ginseng, echinacea, ephedra, green tea extract, and yucca concentrate. In some embodiments, the de-flavored peppercorn may further include a dietary supplement. In some embodiments, the de-flavored peppercorn may be formulated to promote metabolic health. In some embodiments, the peppercorn may be formulated to provide increase metabolic nutrients.

In some embodiments, the de-flavored peppercorn may further comprise at least one or more secondary agents which is a phytonutrient (e.g., isoflavonoids, oligomeric proanthcyanidins, indol-3-carbinol, sulforaphone, fibrous ligands, plant phytosterols, ferulic acid, anthocyanocides, triterpenes, omega 3/6 fatty acids, conjugated fatty acids such as conjugated linoleic acid and conjugated linolenic acid, polyacetylene, quinones, terpenes, catechins, gallates, and quercetin). Sources of plant phytonutrients include, but are not limited to, lecithin, isoflavones, brown rice germ, royal jelly, bee propolis, acerola berry juice powder, Japanese green tea, grape seed extract, grape skin extract, carrot juice, bilberry, flaxseed meal, bee pollen, Ginkgo biloba, primrose (evening primrose oil), red clover, burdock root, dandelion, parsley, rose hips, milk thistle, ginger, Siberian ginseng, rosemary, curcumin, garlic, lycopene, grapefruit seed extract, spinach, and broccoli.

In some embodiments, the de-flavored peppercorn may further comprise an antioxidant. As used herein, the term “antioxidant” refers broadly to a molecule that inhibits the oxidation of other molecules. In some embodiments, the antioxidant is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the de-flavored peppercorn may further comprise a stimulant. As used herein, the term “stimulant” refers to one or more compounds that temporarily increase the rate of body functions. Stimulants can include, for example, amineptine, amiphenazole, amphetamines, bromantan, caffeine, carphedon, ephedrines, fencamfamine, mesocarb, pentylentetrazol, pipradol, salbutamol, salmeterol, terbutaline, and related substances. In some embodiments, the stimulant is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the de-flavored peppercorn may further comprise a lipid. As used herein, the term “lipid” refers to fatty acids and derivatives thereof, as well as substances related biosynthetically or functionally to such compounds. In some embodiments, the lipid is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the flavonoid composition further comprises omega 3 fatty acids. Omega 3 fatty acids can be prepared in powder formulations and can include, for example, eicosapentaenoic acid, docosahexaenoic acid, linolenic acid, hexadecatrienoic acid, stearidonic acid, eicosatrienoic acid, eicosatetraenoic acid, heneicosapentaenoic acid, docosapentaenoic acid, tetracosapentaenoic acid, or tetracosahexaenoic acid, or analogues or derivatives thereof, or combinations thereof. In some embodiments, the omega 3 fatty acid is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the de-flavored peppercorn further comprises one or more pH control agents. In some embodiments, the one or more pH control agents are selected from the group consisting of Acetic acid, Adipic acid, Citric acid, Fumaric acid, Glucono-δ-lactone, Gluconic acid, Lactic acid, Malic acid, Maleic acid, Tartaric acid, Succinic acid, Propionic acid, Ascorbic acid, Phosphoric acid, Sodium orthophosphate, Potassium orthophosphate, Calcium orthophosphate, Sodium diphosphate, Potassium diphosphate, Calcium diphosphate, Pentasodium triphosphate, Pentapotassium triphosphate, Sodium polyphosphate, Potassium polyphosphate, Carbonic acid, Sodium carbonate, Sodium bicarbonate, Potasium carbonate, Calcium carbonate, Magnesium carbonate, Magnesium oxide, or any combination thereof.

In some embodiments, the de-flavored peppercorn may further comprise a flavoring agent. In some embodiments, the flavoring agent may be a flavor selected from berry, strawberry, chocolate, cocoa, lemon, butter, almond, cashew, macadamia nut, coconut, blueberry, blackberry, raspberry, peach, lemon, lime, mint, orange, banana, chili pepper, cinnamon, and pineapple.

In some embodiments, the de-flavored peppercorn may further comprise one or more coating layers. In some embodiments, at least one of the one or more coating layers may be an enteric coating, and in still further embodiments, the core may further include one or more agents selected from solubilizers, stabilizers, colorants, plastizers diluents, fillers, disintegrants, binders, lubricants, surfactants, hydrophobic vehicles, water soluble vehicles, emulsifiers, buffers, humectants, moisturizers, antioxidants, or preservatives. In some embodiments, the core, at least one of the one or more coating layers, or a combination thereof further comprises one or more secondary agents.

In some embodiments, the de-flavored peppercorn further comprises one or more alkaloids. In some embodiments, the one or more alkaloid is selected from piperine, chavicine, isochavicine, isopiperine, capsaicin, vanillyl alcohol, vanillin, vanillic acid, acetovanillon, vanillylmandelic acid, and mohovanillic acid.

An aspect of the disclosure includes a de-flavored peppercorn can be combined with a carrier and provided in any suitable form for consumption by or administration to a subject. In some embodiments, the de-flavored peppercorn is black pepper. In some embodiments, the de-flavored peppercorn is white peppercorn. In some embodiments, the de-flavored peppercorn is green peppercorn. Suitable consumable forms include, but are not limited to, a dietary supplement, food ingredient or additive, a medical food, or nutraceutical.

A food ingredient or additive is an edible substance intended to result, directly or indirectly, in its becoming a component or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or health-beneficial ingredients. A food product according to this invention can, e.g., be in the form of butter, margarine, sweet or savory spreads, condiment, biscuits, nutrition bars, energy bars and cereal bars, bread, cake, cereal, candy, confectionery, soup, milk, yogurt or a fermented milk product, grains, cheese, juice-based, fruit based and vegetable-based beverages, fermented beverages, shakes, flavored waters, teas, coffees, oil, prepared meals, dairy product analogs, eggs and egg product analogs, soups and soup mixes, or any other suitable food.

A dietary supplement is a product taken by mouth that contains a compound of the invention and is intended to supplement the diet. A nutraceutical is a product derived from a food source that provides extra health benefits, in addition to the basic nutritional value found in the food. Dietary supplements and nutraceuticals can be found in many forms such as tablets, coated tablets, pills, capsules, pellets, granules, softgels, gelcaps, liquids, powders, emulsions, suspensions, elixirs, syrup, and any other form suitable for use.

In some embodiments, a de-flavored peppercorn may include a carrier and a preservative to reduce or retard microbial growth. The preservative is added in amounts up to about 5%, preferably from about 0.01% to 1% by weight of the film. Preferred preservatives include sodium benzoate, methyl parabens, propyl parabens, sodium nitrite, sulphur dioxide, sodium sorbate and potassium sorbate. Other suitable preservatives include, but are not limited to, salts of edetate, (also known as salts of ethylenediaminetetraacetic acid, or EDTA, such a disodium EDTA) and natural antimicrobials.

In some embodiments, the de-flavored peppercorn is administered as a nutraceuticals, food additive, or dietary supplements to humans and animals, they can be given per se or as a composition containing, for example, 0.1 to 99% w/w de-flavored peppercorn as an active ingredient in combination with an acceptable carrier. In some embodiments, the composition may comprise 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 99% w/w de-flavored peppercorn, or ranges including, between, and/or spanning the aforementioned values.

### Methods

An aspect of the disclosure includes methods for producing a de-flavored peppercorn. In some embodiments, a method of producing a de-flavored peppercorn comprises contacting a peppercorn with an oil. In some embodiments, the peppercorn is selected from a whole peppercorn, a ground peppercorn, crushed peppercorn, brined peppercorn, and fresh and dried peppercorn. In some embodiments, the peppercorn is black pepper. In some embodiments, the peppercorn is white peppercorn. In some embodiments, the peppercorn is green peppercorn. In some embodiments, the oil is vegetable oil. In some embodiments, the vegetable oil is a sunflower oil, a safflower oil, an olive oil, a soybean oil, a canola oil, and a peanut oil.

In some embodiments, the oil is at a temperature from about 50° C. to about 190° C. In some embodiments, the oil is at a temperature from about 60° C. to about 180° C. In some embodiments, the oil is at a temperature from about 70° C. to about 170° C. in some embodiments, the oil is at a temperature from about 80° C. to about 160° C. In some embodiments, the oil is at a temperature from about 90° C. to about 150° C. In some embodiments, the oil is at a temperature from about 100° C. to about 140° C. In some embodiments, oil is at a temperature equal to or greater than about: 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., or ranges including, between, and/or spanning the aforementioned values.

In some embodiments, the oil and the peppercorn are under pressure. In some embodiments, the pressure is from about 14.5 psi to about 30 psi. In some embodiments, the pressure is equal to or greater than from about 14 psi, 15 psi, 16 psi, 17 psi, 18 psi, 19 psi, 20 psi, 21 psi, 22 psi, 23 psi, 24 psi, 25 psi, 26 psi, 27 psi, 28 psi, 29 psi, 30 psi, or ranges including, between, and/or spanning the aforementioned values.

In some embodiments, the oil is at a pH from about 3 to about 8. The oil pH is equal to or greater than about 3, 4, 5, 6, 7, 8, or ranges including, between, and/or spanning the aforementioned values. In some embodiments, the pH of the oil is adjusted with an acid or a base.

In some embodiments, the peppercorn is agitated before it is contacted with oil. In some embodiments, the peppercorn is agitated in the oil. In some embodiments, the peppercorn is agitated after it is contacted with oil. In some embodiments, the peppercorn is agitated for about 5 minutes, 10 minutes, 15 minutes, 20 minutes, 25 minutes, 30 minutes, 35 minutes, 40 minutes, 45 minutes, 50 minutes, 60 minutes, 70 minutes, 80 minutes, 90 minutes, 100 minutes, 110 minutes, 120 minutes, or ranges including, between, and/or spanning the aforementioned values.

In some embodiments, the peppercorn is fried in oil. In some embodiments, the peppercorn is baked in oil. In some embodiments, the peppercorn is pressure cooked.

In some embodiments, the peppercorn is de-flavored by the reduction of piperine. In some embodiments, the piperine is reduced from the naturally occurring concentration by about 40%, 45%, 50%, 55%, 60%, 65%, 70%, 75%, 80%, 85%, 90%, 95%, 100%, or ranges including, between, and/or spanning the aforementioned values. In some embodiments, the piperine content after de-flavoring is equal to or greater than 0%, 1%, 2%, 3%, 4%, 5%, 10%, 15%, 20%, 25%, 30%, 35%, 40%, 45%, 50%, 55%, 60%, or ranges including, between, and/or spanning the aforementioned values.

In some embodiments, the de-flavored peppercorn retains the metabolic micronutrients. In some embodiments, the de-flavored peppercorn's metabolic micronutrients is equal to or greater than 10 mg/kg, 20 mg/kg, 30 mg/kg, 40 mg/kg, 50 mg/kg, 100 mg/kg, 150 mg/kg, 200 mg/kg, 300 mg/kg, 400 mg/kg, 500 mg/kg, 600 mg, 700 mg/kg, 800 mg/kg, 1000 mg/kg, 1200 mg/kg, 1400 mg/kg, 1600 mg/kg, 1800 mg/kg, 2000 mg/kg, or ranges including, between, and/or spanning the aforementioned values. In some embodiments, the de-flavored peppercorn's metabolic micronutrients is equal to or greater than 0.001%, 0.002%, 0.003%, 0.004%, 0.005%, 0.006%, 0.007%, 0,008%, 0.009%, 0.010%, 0.015%, 0.020%, 0.025%, 0.030%, 0.040%, 0.050%, 0.060%, 0.070%, 0.080%, 0.100%, 0.120%, 0.140%, 0.160%, 0.180%, 0.200% w/w, or ranges including, between, and/or spanning the aforementioned values.

In some embodiments, the oil is separated from the peppercorn after it is contacted with the peppercorn. In some embodiments, the peppercorn is washed after contact with the oil. In some embodiments, the washing is an aqueous solution. In some embodiments, the peppercorn is further dried after the washing. In some embodiments, the peppercorn is further milled.

In aspects, a method of producing a de-flavored peppercorn comprises pretreating the peppercorn. In some embodiments, the peppercorn is black pepper. In some embodiments, the peppercorn is white peppercorn. In some embodiments, the peppercorn is green peppercorn. In some embodiments, the pretreating comprises submerging the peppercorn in a solvent. In some embodiments, the solvent is water or a vegetable oil.

In some embodiments, the pretreating comprises irradiating the peppercorn. In some embodiments, the irradiating is a microwave irradiation. In some embodiments, the microwave irradiation is at 800 MHz, 820 MHz, 840 MHz, 860 MHz, 880 MHz, 900 MHz, 915 MHz, 920 MHz, 940 MHz, 960 MHz, 980 MHz, 1000 MHz, or ranges including, between, and/or spanning the aforementioned values.

In some embodiments, the peppercorn is treated with an enzyme. In some embodiments, the peppercorn is pretreated with an enzyme. In some embodiments, the enzyme is a cellulase, hemicellulose, amylase, pectinase or xylanase.

In some embodiments, the peppercorn is blanched. In some embodiments, the blanching is equal to or greater than 40° C., 45° C., 50° C., 55° C., 60° C., 65° C., 70° C., 75° C., 80° C., 85° C., 90° C., 95° C., 100° C., or ranges including, between, and/or spanning the aforementioned values.

In some embodiments, the peppercorn is treated with an acidic solution. In some embodiments, the acidic solution is a food-grade acid. In some embodiments, the food-grade acid is citric acid, acetic acid, hydrochloric acid, and/or phosphoric acid. In some embodiments, the peppercorn is pretreated with an acidic solution. In some embodiments, the acid solution has a pH from about 2, 2.5, 3, 3.5, 4, 4.5, 5, 5.5, 6.0, 6.5, or ranges including, between, and/or spanning the aforementioned values.

In some embodiments, the peppercorn is treated with ultrasound. In some embodiments, the peppercorn is pretreated with ultrasound. In some embodiments, the ultrasound is equal to or greater than about 15 kHz, 20 kHz, 25 kHz, 30 kHz, 35 kHz, 40 kHz, or ranges including, between, and/or spanning the aforementioned values.

In aspects, a method of extracting piperine from a peppercorn may include contacting the peppercorn with supercritical carbon dioxide. In some embodiments, the supercritical carbon dioxide is at a temperature equal to or greater than about 30° C., 35° C., 40, ° C., 45° C., 50° C., 60° C., 70° C., 80° C., 90° C. or ranges including, between, and/or spanning the aforementioned values. In some embodiments, the method further comprises adjusting pressure. In some embodiments, the adjusting pressure is selected between about 7.4 MPa to about 80 MPa. In some embodiments, the adjusting pressure is selected between 7.4 MPa to about 45 MPa. In some embodiments, the peppercorn moisture content is about 5%, 10%, 15%, 20%, 25%, 30%, or ranges including, between, and/or spanning the aforementioned values. In some embodiments, a co-solvent is used. In some embodiments, the co-solvent is water or ethanol. In some embodiments, the peppercorn is black pepper. In some embodiments, the peppercorn is white peppercorn. In some embodiments, the peppercorn is green peppercorn.

In some embodiments, a method of producing a de-flavored peppercorn includes heating a peppercorn from about 40 to about 50° C., applying approximately 5000 psi of pressure to the peppercorn, wherein the peppercorn comprises approximately 20% w/w moisture, and drying the peppercorn with CO₂.

In aspects, a method of extracting piperine from a peppercorn comprises contacting a peppercorn with a solvent. In some embodiments, the solvent is an organic solvent. In some embodiments, the organic solvent is an alkane solvent or a cycloalkane solvent. In some embodiments, the alkyl solvent is a hexane or a heptane.

In aspects, a method of extracting piperine from a peppercorn comprises contacting a peppercorn with a high intensity light. In some embodiments, the high intensity light is equal to or greater than about 3000 lux, 4000 lux, 5000 lux, 6000 lux, 7000 lux, 8000 lux, 9000 lux, 10000 lux, or ranges including, between, and/or spanning the aforementioned values.

In aspects, a method of extracting piperine from a peppercorn comprises contacting a peppercorn with ultraviolet (UV) radiation. In some embodiments, the UV radiation is equal to or greater than about 150 nm, 200 nm, 250 nm, 300 nm, 350 nm, or ranges including, between, and/or spanning the aforementioned values.

In aspects, a method of reducing the pungency of a peppercorn comprises contacting a peppercorn with an oil. In some embodiments, the peppercorn retains metabolic micronutrients.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the disclosure disclosed herein. Consequently, it is not intended that this disclosure be limited to the specific embodiments disclosed herein, but that it cover all modifications and alternatives coming within the true scope and spirit of the disclosure.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1: Extraction of Piperine Using Vegetable Oils and High Temperature and/or Pressure Treatments

Whole or ground peppercorn was extracted using vegetable oils (sunflower, safflower, olive, soybean, canola, peanut) at temperatures of 135° C., pressures of 30 psi, pH of 5-6 without agitation for 20-40 min. This extraction included frying, pressure cooking, baking, etc. Higher temperatures and pressures helped permeate the cellular matrix of peppercorn, thus enhancing extraction efficiency. Due to its non-polar character, piperine was extracted in the oil phase; metabolic micronutrients, which are lower solubility in non-polar solvents like vegetable oils, were retained in the cellular matrix. The oil was subsequently separated from the insoluble metabolic micronutrients-containing biomass by filtration or centrifugation. Water was used as a process aid to wash the biomass and remove residual oil. Greater than 20% of the metabolic micronutrients were retained in the biomass due to their low solubility in oil and water. Piperine is reduced by >80%, achieving a de-flavored peppercorn product. The washed material was then dried and milled to yield a de-flavored peppercorn powder.

### Example 2: Pre-Treatment Assisted Extraction of Piperine

Whole or ground peppercorn will be pre-treated either as dry solids or while submerged in a solvent such as water, ethanol, or vegetable oil. Pre-treatment methods include microwave irradiation (915 MHz), high pressure processing (100-400 MPa), enzyme treatment (pectinases, cellulases, xylanases etc.), blanching (40-90° C.), acid-based treatment (pH 2-7) and ultrasound (>25 kHz). These pre-treatment methods will be able to alter cellular matrices of plant-based materials including peppercorns, thus improving piperine extraction and bioaccessibility of metabolic micronutrients.

Pre-treated peppercorn will be subsequently extracted with vegetable oil (sunflower, safflower, olive, soybean, canola, peanut) at temperatures from 135° C. and 30 psi, pH of 5-6 without agitation for 20-40 min.

In another extraction, pre-treated peppercorn will be subsequently extracted with either supercritical carbon dioxide, hexane, high intensity light, or UV treatment. Piperine partitions into the oil phase, which will then be separated from the insoluble, metabolic micronutrients-containing biomass by centrifugation or filtering. Water will be used to wash the biomass and remove residual oil. The washed material will be dried and milled (if required) to yield de-flavored peppercorn product.

### Example 3: Supercritical Carbon Dioxide (sCO₂) Extraction of Piperine

A fixed bed of whole or ground peppercorn was contacted with flowing sCO₂ maintained at temperatures of 31.1-90° C. and pressures of 7.4-80 MPa. At these conditions CO₂ acted as a fluid and selectively extracted piperine from the solid biomass while retaining >60% of the original metabolic micronutrients content. Tuning the temperature and pressure within these ranges, sCO₂ flow rate, biomass moisture content, and co-solvent composition yielded de-flavored peppercorn product of varying piperine content (0-60% of the original content), allowing optimization to enhance metabolic micronutrients bioavailability and flavor in the final product.

In a separate study, the majority (>90%) of the piperine was removed in the sCO₂ stream, and subsequently added back to the de-flavored peppercorn product to achieve the ideal ratio of metabolic micronutrients to piperine to enhance bioavailability of metabolic micronutrients. Following piperine extraction, sCO₂ was separated from the biomass by de-pressurizing to atmospheric conditions and allowed residual CO₂ to degas. The biomass was dried and milled to yield the de-flavored peppercorn product. sCO₂ is an attractive non-toxic alternative to harsh chemical solvents such as acetone and hexane for the extraction of non-polar compounds such as piperine.

### Example 4: Extraction in Hexane or Heptane

Whole or ground peppercorn was contacted with hexane or heptane maintained at temperatures of 25-60° C. in an agitated vessel or in a semi-continuous extraction vessel with hexane cross-flow. At these conditions, hexane/heptane selectively extracted piperine from the solid biomass while retaining >60% of the original metabolic micronutrients content. Tuning the temperature, pressure, solvent contact time, and co-solvent composition yielded de-flavored peppercorn product of varying piperine content (0-60% of the original content), allowing optimization to enhance metabolic micronutrients bioavailability in the final product.

In another extraction, the majority of the piperine (>90%) was removed and subsequently added back to the de-flavored peppercorn product to achieve the ideal ratio of metabolic micronutrients to piperine to enhance bioavailability of metabolic micronutrients. Following piperine extraction, hexane/heptane was removed from the biomass by heating the biomass to evaporate the solvent and/or washing with water. The material was then dried and milled.

### Example 5: Isomerization of Piperine to its Non-Pungent Isomers Via High Intensity Light or UV Treatment

Whole or ground peppercorn was exposed to high intensity light (3,000-10,000 lux) or UV radiation (150-350 nm) while being mixed or tumbled in air at room temperature. Light and UV radiation catalyzed the isomerization of trans-trans piperine, the pungent isomer of piperine, to its non-pungent cist-trans, cis-cis, and trans-cis isomers (Friedman et. al., 2007, J. Agric. Food Chem. pp 7131-7139), thus decreasing the pungency of the resulting biomass. Adjusting the light intensity, UV wavelength, and treatment time led to trans-trans piperine reduction of 0-80%, thus allowing for the optimization of the trans-trans piperine quantity in the de-flavored pepper product to promote bioavailability while retaining most of the metabolic micronutrients (>60%) in the original material.

### Example 6: De-Flavored Peppercorn Formulations

The following example demonstrates an embodiment of a de-flavored peppercorn. The de-flavored peppercorn was prepared by the method described in Example 3.

TABLE 1
De-flavored Peppercorn

| Component | % (w/w) |
|---|---|
| Piperine | 0.01-0.6 |
| Carbohydrates | ≥60 |
| Fat | <1 |
| Ash | <10 |
| Protein | 10-15 |
| Metabolic Micronutrients | ≥0.05% |

The prepared de-flavored peppercorn was then combined with a carrier and formulated as a dietary supplement, food ingredient or additive, a medical food, or nutraceutical.

### Example 7: De-Flavored Peppercorn Formulations

The following example demonstrates an embodiment of a de-flavored peppercorn. The de-flavored peppercorn was prepared by the method described in Example 3.

TABLE 2
De-flavored Peppercorn

| Component | | |
|---|---|---|
| Piperine | 0.01-0.6% | w/w |
| Moisture | 5-25% | w/w |
| Metabolic Micronutrients | 0.005 to 0.2% | w/w |

The prepared de-flavored peppercorn was then combined with a carrier and formulated as a dietary supplement, food ingredient or additive, a medical food, or nutraceutical.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ containing,′ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ preferred,“desired,” or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more” to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method of preparing a de-flavored peppercorn, comprising:
contacting a peppercorn with supercritical carbon dioxide at a temperature from about 30 to about 90° C. and a pressure from about 5 to about 80 MPa,
thereby producing a de-flavored peppercorn.

2. The method of claim 1, wherein the temperature is from about 30° C. to about 60° C.

3. The method of claim 1 or 2, wherein the pressure to the peppercorn is from about 7.4 to about 36 MPa.

4. The method of any one of claims 1 to 3, wherein the peppercorn has a moisture content from about 5 to about 25%.

5. The method of any one of claims 1 to 3, further comprising contacting the peppercorn with a solvent.

6. The method of claim 5, wherein the solvent is selected from water, ethanol, or a combination thereof.

7. The method of any one of claims 1 to 5, wherein the de-flavored peppercorn piperine content is about 0% to about 60% of the original piperine content of the peppercorn.

8. The method of any one of claims 1 to 7, wherein the de-flavored peppercorn comprises 0.01% to 6% w/w piperine, 5 to 25% w/w moisture content, and 0.005 to 0.2% w/w metabolic micronutrients.

9. The method of any one of claims 1 to 7, wherein the de-flavored peppercorn comprises 0.01% to about 0.6% w/w piperine, greater than 60% w/w carbohydrates, less than 1% w/w fat, less than 10% w/w ash, about 10 to about 15% w/w protein, and greater than 0.05% w/w metabolic micronutrients.

10. The method of claim 8, wherein the de-flavored peppercorn comprises 0.3% w/w piperine.

11. The method of claim 8 or 10, wherein the metabolic micronutrients comprises at least one of N-trans-caffeoyltyramine, N-trans-feruloyltyramine, and p-coumaroyltyramine.

12. The method of any one of claims 1 to 11, further comprising milling the peppercorn.

13. The method of any one of claims 1 to 12, wherein contacting the peppercorn with supercritical carbon dioxide for 15 minutes to 4 hours.

14. The method of any claims 1 to 13, wherein the peppercorn is selected from a whole peppercorn, a ground peppercorn, and a crushed peppercorn.

15. A dietary supplement, food ingredient or additive, a medical food, or nutraceutical, comprising
a de-flavored peppercorn; and
a carrier,
wherein the de-flavored peppercorn comprises about 0.01% to about 0.6% w/w piperine, greater than 60% w/w carbohydrates, less than 1% w/w fat, less than 10% w/w ash, about 10 to about 15% w/w protein, and greater than 0.05% w/w metabolic micronutrients.

16. The dietary supplement, food ingredient or additive, a medical food, or nutraceutical of claim 15, wherein the carrier is selected from at least one of a sugar, a starch, cellulose, powdered tragacanth, malt, gelatin, talc, excipient, oil, glycol, polyol, ester, agar, buffering agent, alginic acid, isotonic saline, ethyl alcohol, phospholipid, pH buffered solutions, or polyesters.

17. The dietary supplement, food ingredient or additive, a medical food, or nutraceutical of claim 15 or 16, further comprising a preservative.

18. The dietary supplement, food ingredient or additive, a medical food, or nutraceutical of claim 16, wherein the preservative is selected from at least one of sodium benzoate, methyl paraben, propyl paraben, sodium nitrite, sulphur dioxide, sodium sorbate, potassium sorbate or salts of edetate.

19. The dietary supplement, food ingredient or additive, a medical food, or nutraceutical of any one of claims 15 to 18, wherein the metabolic micronutrients comprises at least one of N-trans-caffeoyltyramine, N-trans-feruloyltyramine, and p-coumaroyltyramine.

20. The dietary supplement, food ingredient or additive, a medical food, or nutraceutical of any one of claims 15 to 18, further comprising a secondary agent.

21. The dietary supplement, food ingredient or additive, a medical food, or nutraceutical of claim 20, wherein the secondary agent is selected from a phytonutrient, a flavoring agent, a coating layer, a stabilizer, a colorant, a diluent, a filler, one or more alkaloid, one or more vitamin, or one or more tyramine containing hydroxycinnamic acid amides.

22. The dietary supplement, food ingredient or additive, a medical food, or nutraceutical of claims 15 to 21, wherein the de-flavored peppercorn is formulated to provide increased metabolic nutrients.

23. The dietary supplement, food ingredient or additive, a medical food, or nutraceutical of claims 15 to 21, wherein the de-flavored peppercorn further comprises 5 to 25% w/w moisture content.

24. A method of producing a de-flavored peppercorn, the method comprising:
contacting a peppercorn with one or more oils from about 50 to about 190° C., at about 10 to about 30 psi for about 5 to about 120 minutes,
thereby producing a de-flavored peppercorn.

25. The method of claim 24, wherein the peppercorn is selected from a whole peppercorn, a ground peppercorn, and a crushed peppercorn.

26. The method of claim 24 or 25, wherein the one or more oils is a vegetable oil.

27. The method of claim 26, wherein the vegetable oil is selected from the group consisting of sunflower oil, safflower oil, olive oil, soybean oil, canola oil, and a peanut oil.

28. The method of any one of claims 24 to 27, wherein the oil is at a temperature from about 50° C. to about 135° C.

29. The method of any one of claims 24 to 28, wherein the pressure is from about 14.5 psi to about 30 psi.

30. The method of any one of claims 24 to 29, wherein the oil is at a pH from about 3 to about 8.

31. The method of any one of claims 24 to 30, further comprising agitating the peppercorn for about 5 minutes to about 120 minutes.

32. The method of any one of claims 24 to 31, wherein the peppercorn is fried, baked or pressure cooked.

33. The method of any one of claims 24 to 32, wherein the de-flavored peppercorn comprises greater than 100 mg/Kg of metabolic micronutrients.

34. The method of any one of claims 24 to 33, wherein the de-flavored peppercorn comprises about 0 to about 60% original piperine levels.

35. The method of any one of claims 24 to 34, further comprising separating the vegetable oil from the peppercorn.

36. The method of any one of claims 24 to 35, further comprising washing the peppercorn with an aqueous solution.

37. The method of claim 36, wherein the washing removes residual vegetable oil.

38. The method of any one of claims 24 to 37, further comprising drying the peppercorn.

39. The method of any one of claims 24 to 38, further comprising milling the peppercorn.

40. A method of producing a de-flavored peppercorn, the method comprising:
pretreating a peppercorn,
wherein, pretreating comprises applying pressure to the peppercorn, increasing temperature applied to the peppercorn, irradiating the peppercorn, applying a solvent to the peppercorn, or a combination thereof,
thereby producing a de-flavored peppercorn.

41. A method of extracting piperine from a peppercorn, comprising:
contacting a peppercorn with supercritical carbon dioxide.

42. A method of extracting piperine from a peppercorn, comprising:
contacting a peppercorn with an organic solvent.

43. A method of extracting piperine from a peppercorn, comprising:
contacting a peppercorn with a high intensity light.

44. A method of extracting piperine from a peppercorn, comprising:
contacting a peppercorn with UV radiation.

45. The method of claim 44, wherein the UV radiation is from about 150 nm to about 350 nm.

46. A de-flavored peppercorn produced according to any one of claims 38 to 45.

47. A de-flavored peppercorn, comprising:
0.01% to 0.6% w/w piperine;
5 to 25% w/w moisture content;
0.005 to 0.2% w/w metabolic micronutrients; and
a carrier.

48. The de-flavored peppercorn of claim 47, wherein the de-flavored peppercorn comprises 0.01 to 0.3% piperine.

49. The de-flavored peppercorn of claim 47 or 48, wherein the de-flavored peppercorn comprises 0.3% piperine.

50. The de-flavored peppercorn of claim 47, wherein the moisture content is 25%.
